(19) United States
(12) Plant Patent Application Publication
(10) Pub. No.: US 2009/0229021 P1
(43) Pub. Date: Sep. 10, 2009

(54) Botanical designation rosa hybrid variety denomination 'Poulpah042'

(50) ;

(76) Inventor: Mogens N. Olesen, Fredensborg (DK)

Correspondence Address: POULSEN ROSER PACIFIC, INC., 285 LIBERTY STREET NE, SUITE 370, PMB 153, SALEM, OR, 97301, US

(21) Appl. No.: 12/004,945

(22) Filed: Dec. 20, 2007

(51) Int. Cl. A01H 5/00

(52) U.S. Cl. PLT/120

(57) ABSTRACT

A new miniature rose plant that has abundant, salmon pink flowers and attractive foliage. The variety successfully propagates from softwood cuttings and is suitable for year-round production in commercial glasshouses. This new and distinct variety has shown to be uniform and stable in the resulting generations from asexual propagation.

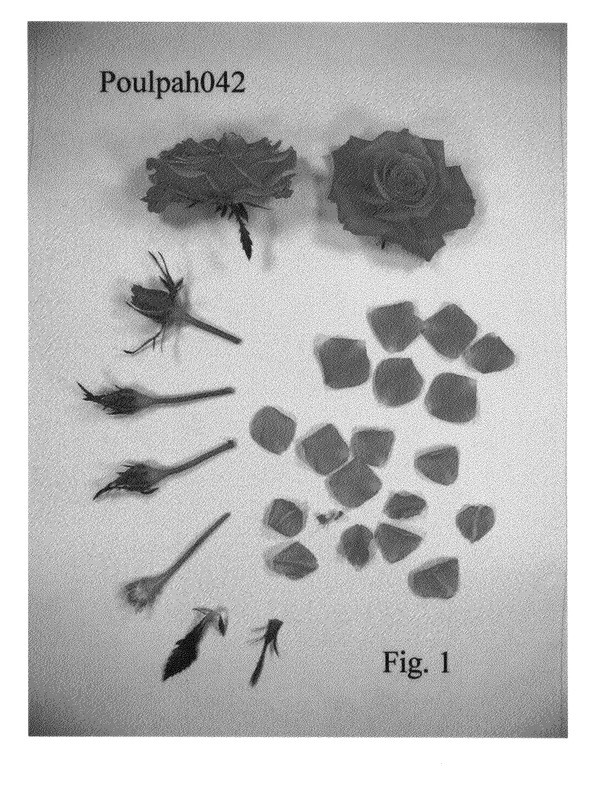

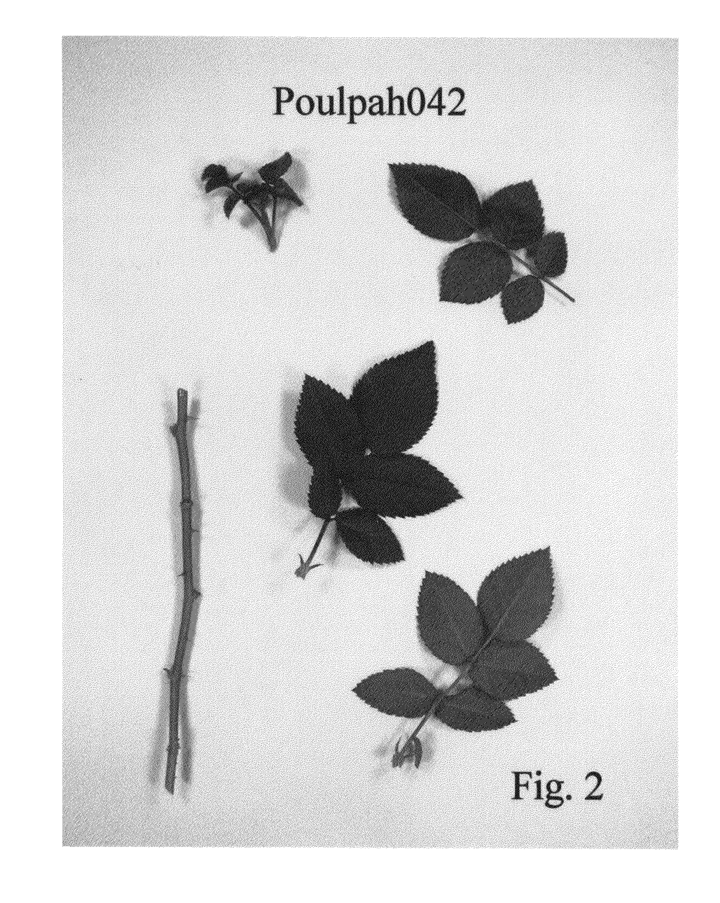

### BOTANICAL DESIGNATION

Rosa hybrid

### VARIETY DENOMINATION ‘Poulpah042’

### SUMMARY OF THE INVENTION

The present invention constitutes a new and distinct variety of miniature rose plant which originated from a controlled crossing between the female seed parent, an unnamed seedling, and the male pollen parent ‘Poulhi013’, U.S. Plant Pat. No. 15,044.

The two parents were crossed during the summer of 2003 and the resulting seeds were planted in a controlled environment in Fredensborg, Denmark. The new variety, named ‘Poulpah042’, originated as a single seedling from the stated cross.

The new variety may be distinguished from its female seed parent primarily by flower color. ‘Poulpah042’ has salmon pink flowers while the seed parent has pink flowers.

The new variety may be distinguished from its male pollen parent primarily by flower color. ‘Poulpah042’ has salmon pink flowers while ‘Poulhi013’ has orange flowers.

The objective of the hybridization of this rose variety for commercial culture was to create a new and distinct variety with unique qualities, such as:
1. Uniform and abundant salmon pink flowers;
2. Vigorous and compact growth;
3. Year-round flowering under glasshouse conditions;
4. Suitability for production from softwood cuttings in pots;
5. Durable flowers and foliage which make a variety suitable for distribution in the floral industry.

This combination of qualities is not present in previously available commercial cultivars of this type, known to the inventor, and distinguish ‘Poulpah042’ from all other varieties of which we are aware.

As part of the rose development program, Mogens N. Olesen germinated the seeds from the aforementioned hybridization and conducted evaluations on the resulting seedlings in a controlled environment in Fredensborg, Denmark. ‘Poulpah042’ was selected by the inventor as a single plant from the progeny of the hybridization in 2003.

Asexual reproduction of ‘Poulpah042’ by cuttings and traditional budding was first done by Mogens N. Olesen in the nursery in Fredensborg, Denmark in 2004. This initial and other subsequent propagations conducted in controlled environments have demonstrated that the characteristics of ‘Poulpah042’ are true to type and are transmitted from one generation to the next.

### BRIEF DESCRIPTION OF THE DRAWING

The accompanying color illustrations show, as true as is reasonably possible to obtain in color photographs of this type, the typical characteristics of the buds, flowers, leaves, and stems of ‘Poulpah042’.

### DETAILED DESCRIPTION OF THE VARIETY

The following is a description of ‘Poulpah042’, as observed in its growth in glasshouses in Fredensborg, Denmark. Observed plants are 3 months of age and were cultivated in 12 cm pots. Color references are made using The Royal Horticultural Society (London, England) Colour Chart, 2001, except where common terms of color are used.

For a comparison, several physical characteristics of the rose variety ‘Poulpah037’, U.S. Plant Pat. No. 17,377. are compared to ‘Poulpah042’ in Chart 1.

CHART 1

| | ‘Poulpah042’ | ‘Poulpah037’ |
|---|---|---|
| Petalage: | 30 to 35 petals, 5 to 8 of | 15 to 20 petals under |
| | which are petaloids. | normal conditions, 5 of |
| | | which are petaloids. |
| Flower Diameter: | 65 to 70 mm | 60 to 90 mm |
| General Tonality of | Red Group 47B | On newly opened flowers |
| Flower Color: | | the flower color is Red- |
| | | Purple Group N57A to |
| | | Red Group C53C. |
| | | Mature flower color |
| | | becomes Red-Purple |
| | | Group N57A to N57C. |

### FLOWER AND FLOWER BUD

Blooming habit: Continuous.
Flower bud:
   Size.—25 to 30 mm length from base of receptacle to end of bud. 8 to 11 mm in diameter.
   Bud form.—Ovate.
   Bud color.—As sepals unfold, petals are Red Group 43B.
   Sepals.—Upper Surface: Color: Yellow-Green Group 147A. Texture: Smooth. Slightly pubescent. Lower Surface: Color: Yellow Green Group 146A. Texture: Smooth. Shape: Apex: Cirrhose. Base: Flat at union with receptacle. Margins: Margins have strong foliaceous appendages on three of the five sepals. Size: 25 to 40 mm long by 5 to 8 mm wide.
   Receptacle.—Surface Texture: Smooth. Shape: Funnel shaped. Size: 5 mm in height by 8 mm wide. Color: Yellow Green Group 144A.
   Pedicel.—Surface: Smooth. Length: 35 to 40 mm average length. Diameter: Normally 2.5 to 3 mm. Color: Yellow-Green Group 144A. Strength: Strong.
   Borne.—Singly.
Flower bloom:
   Fragrance.—Moderate honey scent.
   Duration.—As a pot plant, flowers last from 21 to 28 to days. Petals do not fall cleanly away from plant.
   Size.—Average flower diameter is 65 to 70 mm when open. Depth is generally 30 mm.
   Form.—General shape is a hybrid tea type flower with a tightly closed flower center.
   Shape of flower, side view.—The upper portion is flat while the lower portion is concave.
Petalage: There are normally 30 to 35 petals, 5 to 8 of which are petaloids.
Color:
   Upon opening, petals.—Outermost petals: Upper Surface: Red Group 47C. Lower Surface: Red Group 52B. Innermost petals: Upper Surface: Red Group 50A. Lower Surface: Red Group 52B.
   Basal petal spots.—Upper Surface: Yellow Group 5D. Lower Surface: White Group 155A.
   After opening, petals.—Outermost petals: Upper Surface: Red Group 52A. Occasional streaks the color of White Group 155A and Yellow-Green Group 145A. Lower Surface: Red Group 52B. Occasional streaks the color of White Group 155A and Yellow-Green Group 145A. Innermost petals: Upper Surface: Red Group 52A. Lower Surface: Red Group 52B. Basal petal spots: Upper Surface: Yellow Group 5D. Lower Surface: White Group 155A.
General tonality: On open flower Red Group 47B. After the 10^{th} day, flower tonality becomes Red Group 50B.
Petals:
   Petal reflex.—Weak.
   Petal margin.—Entire. With medium undulations of margin. Point at center of petal margin.
   Shape.—Generally ovate. Base: Rounded. Apex: Cuspidate.
   Size.—Variable. 28 to 40 mm in length by 28 to 40 mm wide.
   Thickness.—Average.
   Texture.—Smooth.
Petaloids:
   Quantity.—Normally, there are 5 to 8 petaloids.
   Size.—22 mm long; 13 mm wide.
   Shape.—Apex is rounded. Base is acute. Generally the petaloids are irregular shape.
   Color.—Upper portion is Red Group 53D. Lower portion is Red 52B.
Reproductive organs:
   Pollen.—None observed.
   Anthers.—Size: 3 to 6 mm long. Color: Yellow-Orange Group 20B. Quantity: 60 to 65.
   Filaments.—Color: Yellow-Orange Group 15C with intonations of Red 41B Length: 6 mm.
   Pistils.—Length: 5 mm long. Quantity: 30 to 35.
   Stigmas.—Slightly inferior relative to the length of the filaments and the height of the anthers. Color: Orange-White Group 159C.
   Styles.—Color: Red Group 46C.
   Seed formation.—Not observed.

### PLANT

Plant growth: Vigorous, compact, upright to bushy. When grown as a 12 cm pot plant on its own roots, the average height of the plant itself is 25 cm and the average width is 20 cm.
Stems:
   Color.—Young wood: Yellow-Green Group 146A. Older wood: Yellow-Green Group 146B.
   Intemodal distance.—Normally 20 mm.
   Length of stems.—From base of stem to flowering portion is generally 18 cm.
   Surface texture.—Young wood: Smooth. Older wood: Smooth.
Prickles:
   Incidence.—8 to 10 per 10 cm of stem.
   Size.—Average length: 3 mm.
   Color.—Juvenile and mature prickles are Greyed-Orange Group 146D.
   Shape.—Concave.
Plant foliage: Normal number of leaflets on normal leaves in middle of the stem: 5 leaflets.
   Compound leaf size.—120 mm (l)×70 mm (w).
   Quantity.—5 leaves per 10 cm of stem.
   Color.—Juvenile foliage: Upper Leaf Surface: Yellow-Green Group 146A. Lower Leaf Surface: Yellow-Green Group 147C. Anthocyanin: Present on undersides of juvenile leaves, coloration is Greyed-Orange Group 176B. Mature foliage: Upper Leaf Surface: Yellow-Green Group 147A Lower Leaf Surface: Yellow-Green Group 148B
Plant leaves and leaflets:
   Stipules.—Size: 12 mm long. Shape: Linear, slightly broad based with outward extending apecies. Margins: Finely serrated with few stipitate glands. Color: Yellow-Green Group 147A.
   Petiole.—Length: 20 mm in length normally. Diameter: Normally 2 mm. Color: Upper surface is Yellow-Green Group 146B with intonations of Greyed-Purple Group 183B. Lower surface is Yellow-Green Group 146B. Underneath: Small prickles observed.
   Rachis.—Size: 45 mm on average. Color: Upper surface is Yellow-Green Group 146B with intonations of Greyed-Purple Group 183B. Lower surface is Yellow-Green Group 146B. Lower Surface: Few small prickles.
   Leaflet.—Size: 45 to 53 mm in length by 22 to 30 mm wide. Margin: Double serrate. General Shape: Elliptical. Apex Shape: Acute. Base Shape: Round. Texture: Smooth. Arrangement: Odd pinnate. Venation: Reticulate. Leaf Gloss: Very glossy.
Disease resistance: Above average resistance to powdery and downy mildew, black spot, and Botrytis under normal growing conditions in Fredensborg, Denmark.

1. A new and distinct variety of rose plant of the miniature class named ‘Poulpah042’, substantially as illustrated and described herein, due to its abundant, salmon pink flowers, vigorous growth, compact habit, suitability for production from softwood cuttings in pots, and durable flowers and foliage that make the variety suitable for distribution in the floral industry.
